(19) United States
(12) Patent Application Publication
(10) Pub. No.: US 2026/0235576 A1
(43) Pub. Date: Aug. 13, 2026

(54) NANOPORE SENSOR DEVICE

(71) Applicant: Oxford Nanopore Technologies PLC, Oxford (GB)

(72) Inventors: Christopher Bohn, Oxford (GB); Nathan Robertson, Oxford (GB); Ângela Alexandra Fragoso Serrano Da Silva, Oxford (GB); Benjamin James Hadwen, Oxford (GB); Peter William Richardson, Oxford (GB); Mark David Jackson, Oxford (GB)

(73) Assignee: Oxford Nanopore Technologies PLC, Oxford (GB)

(21) Appl. No.: 19/159,654

(22) PCT Filed: Feb. 27, 2024
(86) PCT No.: PCT/GB2024/050533

§ 371 Date: Aug. 26, 2025

(30) Foreign Application Priority Data
Feb. 27, 2023 (GB) 2302810.3

(51) Int. Cl. G01N 33/487

(52) U.S. Cl. CPC G01N 33/48721

(57) ABSTRACT

A sensor device (902) for a nanopore sensor, the sensor device (902) comprising: an insulating substrate (903); one or more wells (908) for containing a fluid; wherein the one or more wells (908) are formed on a first side of the substrate (903); a sensor electrode (910) for detecting an ionic current in each of the one or more wells (908); wherein the sensor electrodes (910) are formed on the first side of the substrate (903) at the base of the one or more wells (908); one or more contacts (914) formed on a second side of the substrate (903); and one or more vias (912) extending through the substrate (903); wherein the one or more vias (912) connect the sensor electrodes (910) to the one or more contacts (914); wherein the electrodes (910) comprise a different structure from the one or more contacts (914) and/or the sensor electrodes (910) are made from a different material or materials from the one or more contacts (914).

### RELATED APPLICATIONS

This application is a national stage filing under 35 U.S.C. § 371 of international application number PCT/GB2024/050533, filed Feb. 27, 2024, which claims the benefit of United Kingdom application number GB 2302810.3, filed Feb. 27, 2023, each of which is herein incorporated by reference in its entirety.

### TECHNICAL FIELD

The present invention relates to a nanopore sensor device for use in a nanopore sensing apparatus, in particular to a nanopore sensor device having an insulating substrate.

### BACKGROUND

The use of nanopore devices to sense interactions with molecular entities, for example polynucleotides, is a powerful technique that has been subject to much recent development. Nanopore devices have been developed that comprise an array of nanopore sensing elements, thereby increasing data collection by allowing plural nanopores to sense interactions in parallel, typically from the same sample or analyte.

### SUMMARY OF THE INVENTION

Nanopore devices may typically employ an electrical signal across a nanopore channel to generate a measurement signal that is interpreted to sense and/or characterise molecular entities as they interact with the nanopore. Typically an electrical signal is applied as a potential difference or current across the array of nanopore channels that will provide a meaningful measurement signal to be interpreted. The measurement can include, for example, one of ionic current flow, electrical resistance, or voltage.

An array of sensing elements may be provided on a substrate, which is typically made from a semiconductor such as crystalline silicon. However, forming vias through the substrate to connect to signal processing circuitry, which is typically done by doping the semiconductor, is difficult and expensive. Furthermore, the silicon substrate creates a parasitic capacitance that adds noise to the signal. It will be appreciated that the level of the measurement signal may be sensitive to noise, which may affect the accuracy of sensing and/or characterising the molecular entities being analysed.

It is an aim of the present invention to provide an improved nanopore sensor device.

When viewed from a first aspect the invention provides a sensor device for a nanopore sensing apparatus, the sensor device comprising:
an insulating substrate;
one or more wells for containing a fluid;
   wherein the one or more wells are formed on a first side of the substrate;
one or more sensor electrodes for detecting an ionic current in the one or more wells;
   wherein the one or more sensor electrodes are formed on the first side of the substrate at the base of the one or more wells;
one or more contacts formed on a second side of the substrate; and
one or more vias extending through the substrate;
   wherein the one or more vias connect the one or more sensor electrodes to the one or more contacts;
wherein the one or more sensor electrodes comprise a different structure from the one or more contacts and/or the one or more sensor electrodes are made from a different material or materials from the one or more contacts.

The present invention provides a sensor device for use in a nanopore sensing apparatus. Such a nanopore sensing apparatus may, for example, comprise the sensor device and a detection (for example signal processing) circuit.

The sensor device includes one or more wells formed on one side of an (for example electrically) insulating substrate. The wells are arranged to contain fluid, for example an ionic solution, in which molecular entities to be detected may be provided. The sensor device also includes one or more sensor electrodes, formed at the base of the one or more wells on the same (first) side of the substrate as the wells. Together, the well and the sensor electrode may form a “sensing element”.

One or more vias extend through the substrate, connecting the sensor electrodes with one or more contacts that are formed on the opposite (second) side of the substrate. This allows the measurements obtained at the sensor electrodes to be communicated from the sensor device, for example to a detection circuit.

The sensor electrodes that are formed on one side of the substrate have a different structure and/or are made from a different material from the contacts that are formed on the opposite side of the substrate. This results in the sensor device being asymmetrical, i.e. the sensor electrodes do not mirror the contacts, in structure and/or materials, about the substrate.

It will be appreciated that providing sensor electrodes on one side of the substrate that differ (in structure and/or material) from the contacts on the other side of the substrate allows the sensor electrodes and the contacts to be designed and made so that they are suited to their particular function. This contrasts with conventional substrate manufacturing techniques, in which it is convenient to perform the same processes on either side of the substrate, such that symmetrical features (in both structure and material) are formed.

Furthermore, using a substrate formed from an insulating material helps to reduce the parasitic capacitance of the sensor device. This helps to improve the sensitivity of the measurement and thus the accuracy at which the molecular entities are detected.

The sensor device may be any suitable and desired sensor device for use in a nanopore sensing apparatus. The sensor device may, for example, have a detailed construction as disclosed in WO 2009/077734 or WO 2014/064443, which are herein incorporated by reference in their entireties.

The nanopore sensing apparatus may be any suitable and desired apparatus for sensing molecular entities, for example polynucleotides. The nanopore sensing apparatus may comprise the sensor device and a detection circuit connected to the sensor device.

The detection circuit is preferably arranged to processing the electrical signal(s) output from the (for example sensing elements of the) sensor device, for example as measured at the sensor electrodes. The detection circuit may be arranged to amplify the electrical signal(s) output from the sensor device. The detection circuit may be arranged to (for example control and) apply a bias signal to the (for example sensor electrodes of the) sensing elements, for example to bias the sensor electrodes with respect to one or more reference electrodes. The reference electrode may be a single (for example common) reference electrode or a respective reference electrode for each sensor electrode, for example in the (for example well of the) sensing element. Providing a respective reference electrode for each sensor electrode, for example instead of a single common reference electrode, may help to reduce the cost of the device.

The (for example detection circuit of the) nanopore sensing apparatus may comprise a data processor. The data processor may be implemented in any suitable and desired way, for example the data processor may comprise an application-specific integrated circuit (ASIC), for example configured for nanopore sensing. The design of the (for example data processor of the) detection circuit and its functionality may, for example, be as described in WO 2020/109800, which is herein incorporated by reference in its entirety.

In some embodiments, the (for example data processor of the) detection circuit may be arranged to controlling a potential applied to (for example each of) the sensor electrodes. In some embodiments, the (for example data processor of the) detection circuit may be arranged to measure, digitise and/or output the current (flowing into the wells and as converted by the sensor electrodes).

The sensor device may be connected to the (for example data processor of the) detection circuit in any suitable and desired way. In some embodiments, the nanopore sensing apparatus comprises an interposer, wherein the interposer is connected to the sensor device and to the detection circuit, wherein the interposer is arranged to communicate signals from the sensor device to the detection circuit. The interposer may, for example, comprise a printed circuit board (PCB) or similar component.

The detection circuit may be arranged to output the measured (and, for example, amplified) signals to an analysis system.

The insulating substrate of the sensor device may be formed from any suitable and desired (for example electrically) insulating (non-conducting) material. The insulating substrate may comprise a dielectric substrate. The insulating substrate may be formed from a ceramic material (for example alumina oxide, silicon nitride, quartz), an amorphous solid, a crystal, a non-crystalline material and/or a mineral (for example sapphire or sapphire glass).

In some embodiments the insulating substrate comprises (for example consists of) a glass substrate. Providing a glass substrate helps to reduce the parasitic capacitance of the sensor device, owing to its high electrical resistivity. Glass, through its mechanical properties, also helps to make it easier and cheaper to form vias through the substrate, for example compared to doped vias formed in a semiconductor substrate. This is because vias in a glass substrate may be formed mechanically (for example using a laser) and/or chemically (for example etching).

Any suitable and desired type of glass may be used for the glass substrate. In some embodiments the glass of the substrate comprises a borosilicate glass.

One or more wells for containing a fluid are formed on the first side of the substrate. The wells may be formed and arranged in any suitable and desired way. The sensor device may comprise one or more (for example a plurality of) walls formed on the first side of the substrate, wherein the walls define the one or more wells (i.e. between the one or more walls).

In some embodiments, the sensor device comprises a support structure formed on the first side of the substrate, wherein the support structure defines the (for example one or more walls of the) one or more wells.

The walls and/or the support structure may be formed from any suitable and desired material. The walls and/or the support structure may be formed from an insulating material or a (for example laminated) stack of materials. In some embodiments the walls and/or the support structure are formed as a photoresist structure.

The walls and/or the support structure may be arranged to support a membrane over (for example each of) the wells. The membranes preferably (for example each) contain a nanopore inserted in the membrane. The membrane may comprise amphiphilic molecules such as a lipid or a polymer.

The support structure and the membrane supported over the wells may, for example, take the form as described in WO 2014/064443 and WO 2021/255414, which are herein incorporated by reference in their entireties.

One or more sensor electrodes are formed on the first side of the substrate at the base of (for example each of) the one or more wells. Thus, in some embodiments, a (for example each) well contains (only) a single sensor electrode. The sensor electrodes are arranged (in use of the sensor device) to detect an ionic current in the (for example respective) wells, for example to sense and/or characterise molecular entities as they interact with a nanopore supported by the well.

The sensor electrodes may be arranged at the base of the one or more wells in any suitable and desired way. In some embodiments the sensor electrodes extend over the whole of the base of the (for example respective) wells. The sensor electrodes may extend over an area of the substrate that corresponds to the base of the (for example respective) wells or the sensor electrodes may extend over area of the substrate that is greater than the (area of the) base of the (for example respective) wells, for example such that the sensor electrodes extend underneath the (for example material forming the) walls of the one or more wells.

In some embodiments the sensor electrodes extend partially over the base of the (for example respective) wells. Thus, in some embodiments, the first side of the substrate is exposed at the base of the one or more wells.

The sensor electrodes may have any suitable and desired structure. In some embodiments, (for example each of) the one or more sensor electrodes comprises an electrode base layer proximal to the substrate and an electrode coating exposed to the well.

The electrode base layer may, for example, be provided as a “seed” layer, for example a material that adheres well to the substrate and/or does not disrupt the electrochemical potential of the interface between the solution in the well and the electrode coating.

The electrode coating may be formed from a material that is suited (for example sensitive) to measuring the ionic current and, for example, that adheres well to the electrode base layer.

In some embodiments, the electrode coating at least partially (for example fully) covers the electrode base layer. In some embodiments the electrode coating extends over the whole of the base of the (for example respective) well, for example even though the electrode coating may not necessarily extend over all of the electrode base layer.

The (for example electrode base layer and electrode coating of the) sensor electrodes may be formed from any suitable and desired material. In some embodiments the electrode base layer comprises a transition metal, for example titanium.

In some embodiments the electrode coating has a greater electrical conductivity than the electrode base layer. This may help the electrode coating to be sensitive to measuring the ionic current in the well.

In some embodiments the electrode coating is formed from a less reactive material than the electrode base layer. Forming the electrode coating, which is exposed (during use) to the solution in the well, from a less reactive (for example inert) material than the electrode base layer, helps to reduce any (for example electrochemical) interaction between the electrode coating and the solution in the well, and helps to prevent corrosion of the electrode coating.

In some embodiments the electrode coating comprises a noble metal, for example gold or platinum, and/or a transition metal, for example palladium.

In some embodiments the sensor electrode comprises a silver-silver chloride electrode such as a chloridated silver electrode, for example a silver base layer coated in silver chloride. This type of electrode may be suitable when the ionic solution used in the wells comprises a chloride solution. The silver chloride coating may be formed in-situ in the chloride solution from a silver electrode.

One or more (electrical) contacts are formed on the second (opposite) side of the substrate. The contacts may have any suitable and desired structure. In some embodiments, (for example each of) the one or more contacts comprises a contact base layer proximal to the substrate and a contact outer coating.

The contact base layer may, or example, be provided as a “seed” layer, for example a material that adheres well to the substrate. The contact outer coating may be formed from a material that is suitable for forming an electrical connection, for example to another component in the sensor device or nanopore sensing apparatus. In some embodiments, the contact outer coating at least partially (for example fully) covers the electrode base layer.

The contacts are arranged to be suitable for connecting to other parts (for example components) of the nanopore sensing apparatus. In some embodiments, the contacts are arranged to be make a permanent connection, for example using solder. In some embodiments the contacts may be shaped (for example comprise a depression) for receiving a solder ball.

In some embodiments, the contacts are arranged to be make a temporary (removable) connection, for example using a spring contact. In some embodiments the contacts may be shaped (for example comprise a protrusion) for receiving a sprung contact.

The (for example contact base layer and contact outer coating of the) contacts may be formed from any suitable and desired material. In some embodiments the contact base layer comprises a transition metal, for example copper and/or titanium.

In some embodiments, the contact outer coating comprises a noble metal, for example gold.

In some embodiments, the contact outer coating has a greater electrical conductivity than the contact base layer. This may help the contact outer coating to form a good electrical connection, for example to another component in the sensor device or nanopore sensing apparatus.

In some embodiments, the one or more contacts comprises a contact intermediate layer between the contact base layer and the contact outer coating. This may help to form the shape of the contacts and to form an effective connection between the contact base layer and the contact outer coating.

The contact intermediate layer may comprise any suitable and desired material. In some embodiments, the contact intermediate layer comprises a transition metal, for example nickel or palladium. Nickel may be used when the contacts are arranged to be make a permanent connection. Palladium may be used when the contacts are arranged to be make a temporary connection.

The contact intermediate layer and the contact outer layer may be in the form of an electroless nickel immersion gold (ENIG) or electroless palladium immersion gold (EPIG) finish.

In some embodiments, the contact outer coating has a greater electrical conductivity than the contact intermediate layer. Again, this may help the contact outer coating to form a good electrical connection, for example to another component in the sensor device or nanopore sensing apparatus.

In some embodiments, the sensor device comprises an insulating layer on the second side of the substrate. The insulating layer may at least partially (for example substantially fully) surround the one or more contacts. The insulating layer may be in contact with the second side of the substrate. When the sensor device comprises a plurality of contacts, the insulating layer may be arranged between the contacts.

The insulating layer may comprise and suitable and desired insulating material, for example silicon dioxide, a fluoropolymer or a thermopolymer such as polybenzoxazole (PBO).

The sensor device has one or more vias that extend through the substrate, i.e. from the first side to the second side of the substrate. The sensor electrodes are connected to the one or more contacts (for example each sensor electrode is connected to a respective contact) by one or more of the (for example respective) vias.

The vias may be arranged in any suitable and desired way. In some embodiments, the via is defined by a wall through the substrate.

In some embodiments, the one or more vias (for example each) comprise a conductor forming a conductive path through the via. The conductor forming a conductive path through the via may be arranged in any suitable and desired way in the via to form the conductive path. In some embodiments, the conductor extends through the via. In some embodiments, the conductor comprises a conducting barrel that at least partially lines the (for example wall of the) via.

In some embodiments, a material is arranged (for example in each via) to retain the conductor in the via. The material arranged to retain the conductor in the via may be any suitable and desired material. In some embodiments, the material arranged to retain the conductor in the via is different from a material of the conductor.

In some embodiments, the material arranged to retain the conductor in the via substantially fills the via. In some embodiments, the material arranged to retain the conductor in the via comprises a sealant, for example an adhesive. Preferably, the material arranged to retain the conductor in the via is arranged to substantially seal the via (for example between the first side of the via and the second side of the via). In some embodiments, when the conductor comprises a conducting barrel that at least partially lines the (for example wall of the) via, the material arranged to retain the conductor in the via substantially fills the conducting barrel. Preferably, the material arranged to retain the conductor in the via is arranged to substantially seal the conducting barrel in the via.

The (for example each) of the one or more vias may be arranged to connect the sensor electrode to the (for example respective) contact in any suitable and desired way. In some embodiments, the one or more vias (for example each) comprise a conducting cap at one or both ends of the via. When the vias comprise a conducting cap, preferably the conducting cap is connected to the (respective) sensor electrode and/or to the (respective) contact. Thus, the conducting cap may be connected to the (respective) electrode base layer and/or the (respective) contact base layer.

### DESCRIPTION OF THE FIGURES

Certain preferred embodiments of the invention will now be described, by way of example only, with reference to the accompanying drawings, in which:

FIG. 1 shows a schematic diagram of a nanopore array device;

FIG. 2 shows a schematic cross-sectional view of part of a nanopore array device;

FIG. 3 shows a schematic cross-sectional view of various components of a nanopore sensor in accordance with an embodiment of the present invention;

FIG. 4 shows a schematic cross-sectional view of a sensor device in accordance with an embodiment of the present invention;

FIG. 5 shows a schematic cross-sectional view of an electrode on the front side of a substrate in accordance with an embodiment of the present invention;

FIG. 6 shows a schematic cross-sectional view of a sensor device in accordance with an embodiment of the present invention;

FIG. 7 shows a plan view of the electrode shown in FIG. 6;

FIG. 8 shows a schematic cross-sectional view of an electrode on the front side of a substrate in accordance with an embodiment of the present invention;

FIG. 9 shows a schematic cross-sectional view of a sensor device in accordance with another embodiment of the present invention; and

FIG. 10 shows a schematic cross-sectional view of a substrate contact on the back side of a substrate in accordance with an embodiment of the present invention.

### DETAILED DESCRIPTION

Various embodiments of the present invention will now be described in the context of nanopore sensors, which may be used for the sensing of molecular entities.

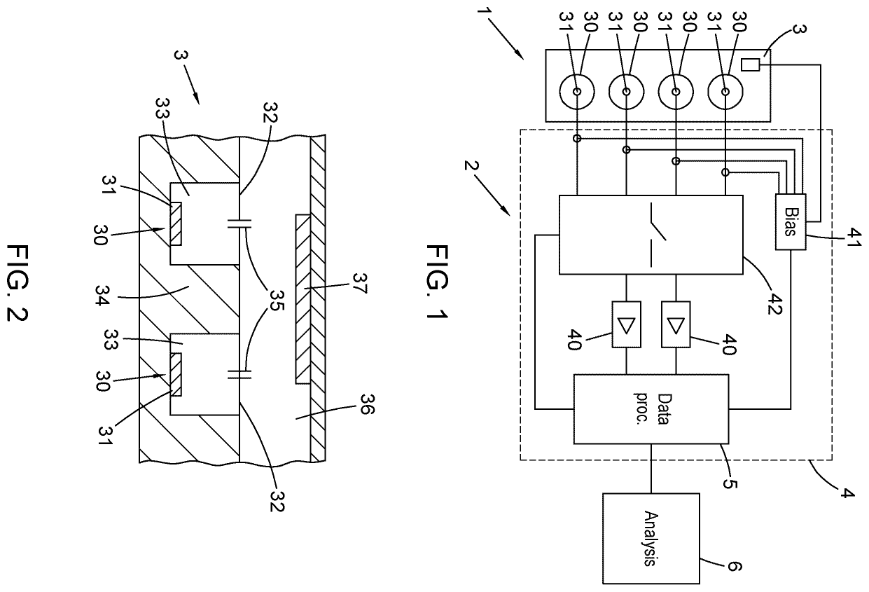

FIG. 1 shows a schematic diagram of a nanopore array device 1 for sensing interactions of a molecular entities. The nanopore array device 1 comprises a sensing apparatus 2 comprising a sensor device 3 and a detection circuit 4 that is connected to the sensor device 3.

The sensor device 3 comprises an array of sensing elements 30 that each support respective nanopore channels that are capable of an interaction with a molecular entity. The sensing elements 30 comprise respective sensor electrodes 31. In use, each sensing elements 30 outputs an electrical measurement at its sensor electrode 31 that is dependent on an interaction of a molecular entity with the nanopore. The sensor device 3 is illustrated schematically in FIG. 1 but may have a variety of configurations, some non-limiting examples being as follows.

In one example, the sensor device 3 may have the form shown in FIG. 2, which shows a schematic cross-sectional view of the sensor device 3. As shown in FIG. 2, the sensor device 3 comprises an array of sensing elements 30. Each sensing element 30 comprises a membrane 32 supported across a well 33 formed in a support structure 34. A nanopore 35 is inserted in the membrane 32 across each well 33 that provides a channel extending from one side of the membrane to the other. The membrane 32 may comprise amphiphilic molecules such as a lipid or a polymer.

Each membrane 32 seals the respective well 33 from a liquid sample (“cis”) chamber 36, which extends across the array of sensing elements 30 and is in fluid communication with each nanopore 35. Each well 33 has a sensor electrode 31 at the base of the (“trans”) well 33. A common electrode 37 is provided in the sample chamber 36 for providing a common reference signal (typically a potential or voltage) to each sensing element 30. In some embodiments, a respective reference electrode for each sensor electrode, for example in the well of the sensing element, may be provided. A liquid sample may be provided in each of the wells.

In use, the sample chamber 36 receives a sample containing an ionic solution and molecular entities which interact with the nanopores 35 of the sensing elements 30. In a sensing mode of the sensing apparatus 2, an ionic current flows from the common electrode 37 to sensor electrodes 31 through the respective nanopores 35. Molecules within (for example passing through) the nanopores 35 restrict the flow of ions through the nanopores and thus modulate the ionic current measured by the sensor electrodes 31 over time. This may then be used to characterise (for example identify) the molecules.

Two sensing elements 30 are shown in FIG. 2 for clarity, but in general any number of sensing elements 30 may be provided. Typically, a large number of sensing elements 30 may be provided to optimise the data collection rate, for example 256, 1024, 4096 or more sensing elements 30.

The sensor device 3 may, for example, have a detailed construction as disclosed in WO 2009/077734 or WO 2014/064443, which are herein incorporated by reference in their entireties.

The nanopore channels 35 and associated elements of the sensing elements 30 may be as follows, without limitation to the example shown in FIG. 2.

The nanopore channel 35 is a pore, typically having a size of the order of nanometres. In embodiments where the molecular entities are polymers that interact with the nanopore channel 35 while translocating therethrough in which case the nanopore channel 35 is of a suitable size to allow the passage of polymers therethrough.

The use of nanopores for characterising molecular entities is well-known in the art. Exemplary nanopores for use in the invention include a protein pore, an origami pore and a solid-state pore. The protein pore may be a wild type or modified. The transmembrane pore may be derived from or based on for example Msp, alpha-hemolysin (α-HL), lysenin, CsgG, ClyA, Sp1 and haemolytic protein fragaceatoxin C (FraC). Examples of materials in which solid state pores may be provided are graphene and silicon nitride. The dimensions of the pore may be such that only one polymer may translocate the pore at a time.

The arrangement of the detection circuit 4 shown in FIG. 1 will now be discussed. The detection circuit 4 is connected to the electrodes 31 of each sensing element 30 and has the primary function of process the electrical signals output therefrom. The detection circuit 4 also has the function of controlling the application of bias signals to each sensing element 30.

The detection circuit 4 includes plural detection channels 40. Each detection channel 40 receives an electrical signal from a single sensor electrode 31 and is arranged to amplify that electrical signal. The detection channel 40 is therefore designed to amplify very small currents with sufficient resolution to detect the characteristic changes caused by the interaction of interest. The detection channel 40 is also designed with a sufficiently high bandwidth to provide the time resolution needed to detect each such interaction. These constraints require sensitive and therefore expensive components.

Each detection channel 40 may be similar to standard single channel recording equipment as describe in Stoddart D et al., Proc Natl Acad Sci USA. 2009 May 12; 106(19): 7702-7, Lieberman K R et al., J Am Chem Soc. 2010 Dec. 22; 132(50): 17961-72 and WO 2000/28312, which are herein incorporated by reference in their entireties. Alternatively, each detection channel 40 may be arranged as described in detail in WO 2010/122293, WO 2011/067559 or WO 2016/181118, which are herein incorporated by reference in their entireties.

The analyte of interest to be detected by the nanopore may be a polynucleotide such as DNA or RNA. The analyte may be a polypeptide or a polysaccharide. The number of sensing elements 30 in the array is greater than the number of detection channels 40 and the nanopore array device is operable to take measurements of a polymer from sensing elements 30 selected in a multiplexed manner, in particular an electrically multiplexed manner. This is achieved by providing a switch arrangement 42 between the sensor electrodes 31 of the sensing elements 30 and the detection channels 40.

For clarity, FIG. 1 shows a simplified example with four sensing elements 30 and two detection channels 40, but the number of sensor cells 30 and detection channels 40 is typically much greater. For example, for some applications, the sensor device 3 may comprise a total of 4096 sensing elements 30 and 1024 detection channels 40. The switch arrangement 42 may be arranged as described in detail in WO 2010/122293. For example, the switch arrangement 42 may comprise plural 1-to-N multiplexers each connected from a detection channel 40 to a group of N sensing elements 30 and may include appropriate hardware such as a latch to select the state of the switching.

By switching of the switch arrangement 42, the nanopore array device 1 may be operated to amplify electrical signals from sensing elements 30 selected in an electrically multiplexed manner. The detection circuit 4 includes a data processor 5 which receives the output signals from the detection channels 40. The data processor 5 acts as a controller that controls the switch arrangement 42 to connect detection channels 40 to respective sensing elements 30, as described further below.

In addition, the detection circuit 4 includes a bias control circuit 41 to perform the function of controlling the application of bias signals to each sensing element 30. The bias control circuit 41 is connected to the common electrode 37 and to the sensor electrodes 31 of each sensing element 30. The bias signals are selected to bias the sensor electrodes 31 with respect to common electrode 37 to control translocation of the molecular entities with respect to the nanopores 35. In general, it would be possible for a bias signal supplied to a given sensing element 30 to be a drive bias signal that causes translocation to occur at the sensing element 30 or an inhibition bias signal that inhibits translocation to occur at the sensing element 30.

The bias control circuit 41 is controlled by the data processor 5. The data processor has a mode of operation for the bias control circuit 41. Namely, three independent test bias signals are supplied to all the sensing elements 30, thereby causing ionic current flow from the common electrode 37 through the nanopores 35 to the sensor electrodes 31 of each sensing elements 30. The corresponding current flow for each test signal is recorded in the data processor 5 as an amplified electrical signal.

The data processor 5 is arranged as follows. The data processor 5 is connected to the output of the detection channels 40 and is supplied with the amplified electrical signals therefrom. The data processor 5 stores and analyses the amplified electrical signals from the test bias signals to create a calibrated signal. The data processor 5 also controls the other elements of the detection circuit, including control of the bias voltage circuit 41 as described above and control of the switch arrangement 42 as described below. The data processor 5 forms part of the detection circuit 4 and may be provided in a common package therewith, in some examples on a common circuit board. The data processor 5 may be implemented in any suitable form, for example as a processor running an appropriate computer program or as an ASIC (application specific integrated circuit).

The data processor 5 of the nanopore array device 1 is connected to an analysis system 6. The data processor 5 also supplies the amplified output signals to the analysis system 6. The analysis system 6 performs further analysis of the amplified electrical signal which is a raw signal representing measurements of the property measured at the nanopore. Such an analysis system 6 may, for example, estimate the identity of the molecular entity in its entirety or, in the case that the molecular entity is a polymer, may estimate the identity of the polymer units thereof. Thus, the analysis system may be configured as a computer apparatus running an appropriate program. Such a computer apparatus may be connected to the data processor 5 of the nanopore array device 1 directly or via a network, for example within a cloud-based system.

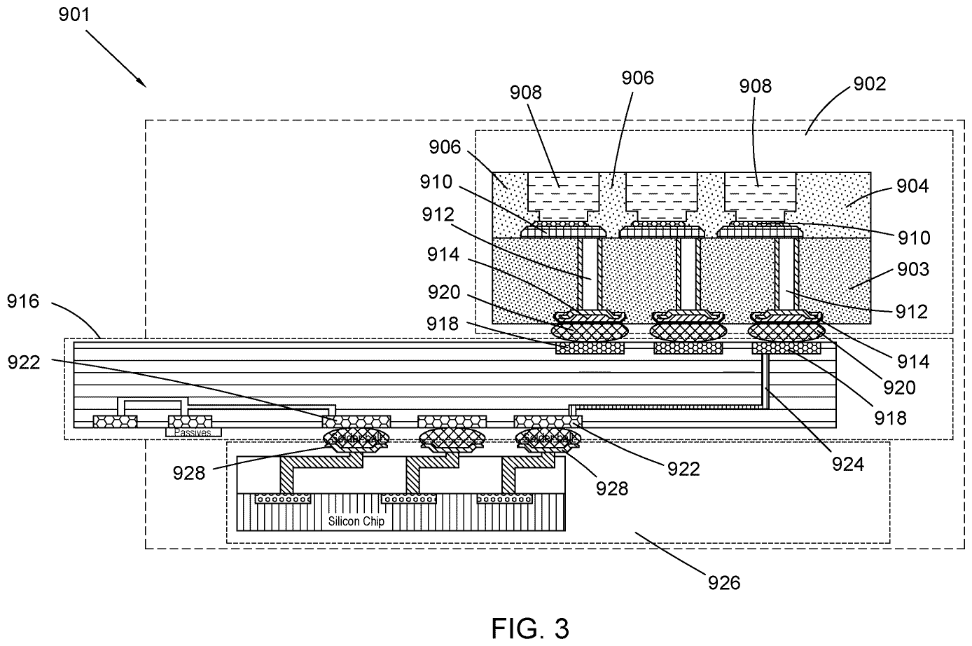

FIG. 3 shows schematically in cross-section various components of a sensing apparatus 901 in accordance with an embodiment of the present invention. The sensing apparatus 901 includes a nanopore sensor device 902 comprising a substrate 903 on which is formed a support structure 904. The sensor device 902 may, for example, be used as the sensor device 3 in the nanopore array device 1 shown in FIGS. 1 and 2.

The support structure 904 comprises a plurality of walls 906 that define between them a plurality of wells 908. Although only a cross-section is shown in FIG. 3, it will be appreciated that a sensor device 902 may comprise a two dimensional array of wells 908 distributed over the substrate 903.

The support structure 904 is formed on the “front” side of the substrate 903. The support structure 904 is configured to support a membrane over each of the wells 908, with the membrane being designed to contain a nanopore inserted in the membrane. The support structure 904 and the membrane supported over each of the wells 908 may, for example, take the form as described in WO 2014/064443 and WO 2021/255414, which are herein incorporated by reference in their entireties.

The wells may be filled with an ionic (for example aqueous) solution. The ionic solution may comprise a soluble electrode mediator, for example ferricyanide or ferrocyanide. The ionic solution may be as described in WO 2018/060740, which is hereby incorporated by reference in its entirety.

A plurality of sensor electrodes 910 are also formed on the front side of the substrate 903, such that a sensor electrode 910 is provided at the bottom of each well 908.

Each sensor electrode 910 may be used to facilitate measurement of the ionic current between the common electrode to the sensor electrode 910 in the respective well 908.

The sensor electrode 910 may be formed from a material appropriate to create an electrochemical interface and dependent on the chemistry used. Examples include platinum (for example for use with soluble redox couple such as a ferri/ferrocyanide mediator) and silver-silver chloride (for example for used with a chloride ionic solution), for example as described in Ayub M et al, Electrochimica Acta 55 (2010) 8237-8243, which is herein incorporated by reference in its entirety.

A plurality of vias 912 are formed through the substrate 903 to connect the respective plurality of sensor electrodes 910 to a respective plurality of substrate contacts 914 on the opposite “back” side of the substrate 903. The vias 912 each have a diameter, for example chosen in accordance with the pitch between the wells. In an exemplary geometry, the distance between (“pitch” of) the wells (and thus also between the plurality of sensor electrodes 910 and between the plurality of substrate contacts 914) may be in the range 100 μm to 300 μm, for example approximately 200 μm. The diameter of the vias 912 may be the range 40 μm to 100 μm, for example approximately 70 μm. The (for example circular) sensor electrodes 910 may have a diameter in the range 45 μm to 105 μm, for example approximately 90 μm.

The sensing apparatus 901 also typically includes an electrical interposer. This is typically a printed circuit board (PCB) 916, but could also be a “PCB-like” technology, for example a ceramic interposer, High Density PCB (HD-PCB), or so-called “substrate PCB”. The PCB 916 comprises a plurality of input contacts 918 that are connected to the plurality of substrate contacts 914 respectively formed on the substrate 903. Such a connection may be permanent, for example as made by a permanently adhered electrical contact, for example a ball of solder 920 (as shown in FIG. 3), a thermally bonded metal (for example Cu) pillar or an anistropic conductive film (ACF) tape. Alternatively, the connection may be designed to be broken and re-made many times, for example by using a mechanical spring contact, or similar.

The PCB 916 also comprises a plurality of output contacts 922. The plurality of output contacts 922 are connected to the plurality of input contacts 918 by a plurality of tracks 924 formed on the PCB 916.

The sensing apparatus 901 further includes an application-specific integrated circuit (ASIC) 926, designed for nanopore sensing. Further details of typical ASIC designs and functionality may be found in WO 2020/109800, which is herein incorporated by reference in its entirety. In summary, the ASIC performs the functions of:
1. Controlling the potential applied to each of the electrodes 910 in the array; and
2. Measuring, digitising and outputting the current flowing into plurality of wells 908 and as converted by the plurality of electrodes 910.

The ASIC 926 comprises a plurality of ASIC contacts 928 for connecting to the plurality of output contacts 922 on the PCB 916 respectively. It will be appreciated that, in some embodiments, the PCB may be omitted and the ASIC 926 connected to the sensor device 902 directly.

Thus the plurality of sensor electrodes 910 in the plurality of wells 908 are connected to the ASIC 926 via the plurality of vias 912 through the substrate 903, the plurality of substrate contacts 914 on the opposite side of the substrate 903, the plurality of input contacts 918 on the PCB 916, the plurality of tracks 924 on the PCB 916, the plurality of output contacts 922 on the PCB 916 and the plurality of ASIC contacts 928. The PCB 916 is provided to match up and connect the layout of the plurality of substrate contacts 914 (corresponding to the layout of the plurality of sensor electrodes 910 and the plurality of wells 908) to the plurality of ASIC contacts 28.

The PCB 916 may also comprise contact pads for other circuit components, connections between the circuit components and the outputs of the ASIC 926, and the outputs for connection to the rest of the nanopore array device. The PCB 916 may also house other electronic components, thus forming a “disposable” part of the sensor device 902. These may include de-coupling capacitors, test points for test features, non-volatile memory for storing identification and/or calibration parameters, components for regulating the temperature of the sensor device 902 (for example resistors for Joule heating and/or a temperature sensor), analogue circuits for supporting operation of the ASIC 926 (for example precision voltage references), etc. In some embodiments, one or more of these components may be provided by (for example integrated into) the ASIC 926.

The PCB 916 may also be arranged to supply a potential to the common (reference) electrode of the sensor device. This may be via a (for example platinum) connecting wire to an external reference electrode in contact with the liquid in the “cis” volume, or via a common electrode disposed on the sensor device 902 and connected through a via. In embodiments in which separate reference electrodes (for example for each well) are provided, the PCB 916 may supply a potential to these reference electrodes in a similar manner, for example through respective wires or vias.

Although only a single nanopore sensor device 902 (having a single substrate 903) is shown, it will be appreciated that any number of nanopore sensor devices 902 (and associated substrates 903) may be provided in the sensing apparatus 901, for example each having multiple sensing elements. Multiple nanopore sensor devices 902 may, for example, be connected to the same ASIC 926, for example via the same PCB 916.

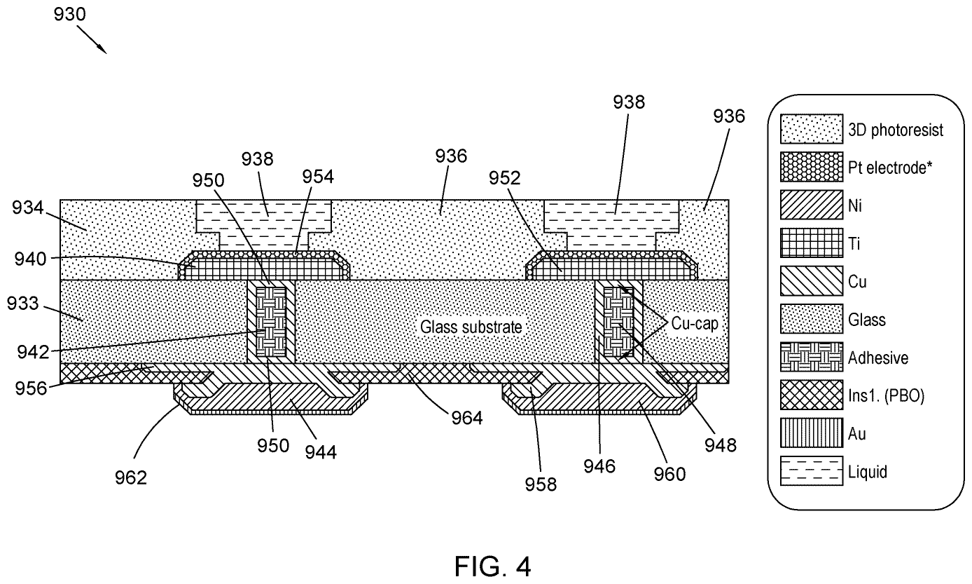

FIG. 4 shows schematically in cross-section a sensor device 930 in accordance with an embodiment of the present invention. The sensor device 930 may, for example, be used in the nanopore sensing apparatus 901 shown in FIG. 3.

Similarly to the sensor device shown in FIG. 3, the sensor device 930 shown in FIG. 4 comprises a substrate 933 on which is formed a support structure 934 having a plurality of walls 936 that define between them a plurality of wells 938. The support structure 934 is formed on the front side of the substrate 933 and is configured to support a membrane over each of the wells 938, with the membrane being designed to contain a nanopore inserted in the membrane.

A sensor electrode 940 is formed on the front side of the substrate 933 at the bottom of each well 938. A plurality of vias 942 are formed through the substrate 933 to connect the respective plurality of sensor electrodes 940 to a respective plurality of substrate contacts 944 on the back side of the substrate 933.

In the embodiment shown in FIG. 4, the substrate 933 is formed from glass. Glass has a number of physical properties that make it a suitable material for this application, for example:
a. high electrical resistivity;
b. its mechanical properties: good rigidity, flatness, resistance to fracture;
c. good thermal conductivity, supporting good thermal communication between the ASIC and the wells, which may be important for controlling the temperature of liquid in the wells;
d. a coefficient of thermal expansion that is tolerably well matched to that of the PCB and the (typically) photoresist material used to form the well structure;
e. its compatibility with manufacturing processes used to make vias through the structure;
f. being manufacturable in volume and at scale; and
g. a low potential to introduce chemical or biochemical contamination into the liquid well.

An example of a suitable glass material is borosilicate, for example Asahi Glass AN100, AN Wizus, Corning 1737, Corning 7740, Schott Borofloat 33.

In some embodiments, the substrate may be formed from insulating materials, including, for example, ceramic materials (for example alumina oxide, silicon nitride, quartz), amorphous solids or non-crystalline materials.

The support structure 934 is formed on the substrate 933 as a photoresist structure, in order to form the plurality of wells 938. The support structure 934 may be formed from an insulating material (for example as a photoresist structure) or a (for example laminated) stack of materials.

The support structure 934 may, for example, take the form as described in WO 2014/064443 and WO 2021/255414, which are herein incorporated by reference in their entireties.

The vias 942 through the substrate 933 each comprise a conductive (for example copper) barrel coating 946 that is filled with adhesive 948 to hermetically seal the vias 942. The vias 942 each have a diameter of approximately 70 μm. The pitch between the vias 942 (and thus also between the plurality of sensor electrodes 940 and between the plurality of substrate contacts 944) is approximately 200 μm. Optionally, a copper cap 950 is provided at each end of the vias 942 (proximate to each of the front and back sides of the substrate 933), which forms a flat surface on which the sensor electrodes 940 and the substrate contacts 944 may be formed.

The sensor electrodes 940 on the front side of the substrate 933 are formed of two layers: a base layer 952 made of titanium and a coating 954 made of platinum. The titanium base layer 952 adheres well to the substrate 933 and thus seals the non-inert copper caps 950 from the liquid in the respective wells 938. The platinum coating 954 provides an inert layer of good conductivity for measuring the ionic current in the well 938.

The sensor electrodes 940 shown in FIG. 4 are substantially circular, with a diameter of approximately 90 μm. The sensor electrodes 940 are arranged substantially symmetrically with respect to the respective wells 938 and to the respective vias 942, i.e. each set of the well 938, the sensor electrode 940 and the via 942 are substantially coaxial with each other. This contrasts with the arrangement shown in FIG. 3 (and other embodiments), in which the sensor electrodes 910 are offset from (and not coaxial with) the respective vias 912 and the wells 908 are offset from the centre of the (titanium base layer) of the respective sensor electrodes 910.

Furthermore, as shown in FIG. 4, the platinum coating 954 fully covers the titanium base layer 952, such that the platinum coating 954 extends around the sides of the titanium base layer 952. The base layer 952 helps adhere the sensor electrode 940 to the substrate 933.

In contrast, as shown in FIG. 3, the sensor electrodes 910 have a platinum coating that has a smaller diameter than the respective titanium base layer, and the platinum coating is offset from the centre of the titanium base layer.

Thus, in the embodiment shown in FIG. 3, the platinum coating does not fully cover the titanium base layer, although the platinum coating does fully extend over the base of the well 908. This means that liquid in the well is only exposed to the platinum coating of the sensor electrode and not to the titanium base layer. It also creates a (lateral) separation between the copper in the vias 912 and the liquid in the respective wells 908, which is better electrochemically for the wells 908 and the measurement of the ionic currents.

In some embodiments, the sensor electrode 940 may not fully extend over the base of the well 908. Thus some of the liquid in the well may be exposed to the substrate 930.

The substrate contacts 944 on the back side of the substrate 933 are each formed of four layers: a base layer 956 made of copper, a first (proximal) intermediate layer 958 made of copper, a second (distal) intermediate layer 960 made of nickel and an outer coating 962 made of gold. second (distal) intermediate layer 960 and the outer coating 962 may be applied to the first (proximal) intermediate layer 958 using an electroless nickel immersion gold (EPIG) finish. The copper base layer 956 connects to, and extends on the substrate 933 over and around, the copper cap 950 on the via 942. The copper base layer 956 provides a platform on which the other layers of the substrate contact 944 are based. It will be appreciated that this structure is exemplary and there may be other suitable structures for the substrate contacts 944.

An insulation layer 964 (for example made of polybenzoxazole (PBO)) may be provided over the copper base layers 956, with openings in the insulation layer 964 to allow contact between each copper intermediate layer 958 and the respective copper base layer 956. The copper intermediate layer 958 extends over a portion of the insulation layer 964, for example forming a recess in the centre of the copper intermediate layer 958. The nickel intermediate layer 960 extends over all of the copper intermediate layer 958. The gold outer coating 962 covers all of the nickel intermediate layer 960.

It will be appreciated that the structure of the substrate contacts 944 may differ, for example depending on the configuration of the connection, for example to a PCB or ASIC, as well as if the connection is permanent (for example using solder) or temporary (for example via a spring contact).

The substrate contacts 944 on the back side of the substrate 933 are configured for connection to input contacts of a PCB by respective balls of solder (for example as shown in FIG. 3). It will be seen that the substrate contacts 944 on the back side of the substrate 933 are different in configuration and materials from the sensor electrodes 940 on the front side of the substrate 933. This asymmetry in the design of the sensor device 930 enables the sensor electrodes 940 (for measuring the ionic current in the wells 938) and the substrate contacts 944 (for connecting to the PCB contacts) to be designed to be suited to their respective functions.

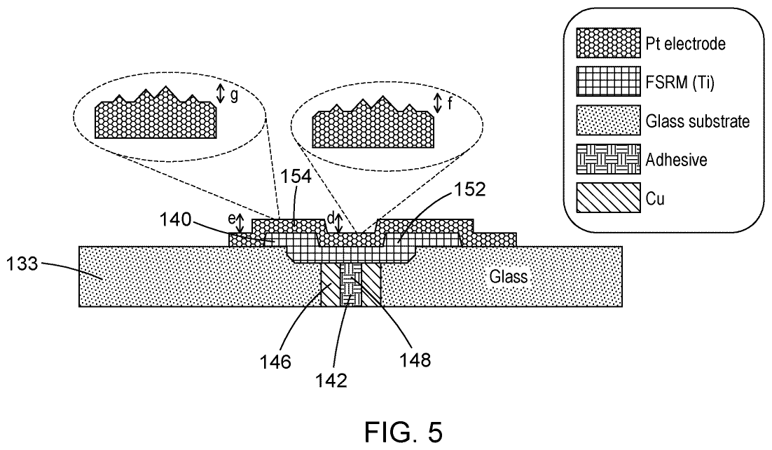

FIG. 5 shows an embodiment of a sensor electrode 140 on the front side of a substrate 133, similar to the embodiment shown in FIG. 4. Only the upper half (showing the front side) of the substrate 133 is shown in FIG. 5.

In this embodiment, in which the sensor electrode 140 is centred on the via 142, the via 142 comprises a copper barrel coating 146 that is filled with adhesive 148, but the via 142 does not have a copper cap. Instead, the front side of the substrate 133 surrounding the via 142 is recessed and the titanium base layer 152 of the sensor electrode 140 is formed in and around the recess. The platinum coating 154 fully covers the titanium base layer 152 and thus follows the recessed profile of the titanium base layer 152.

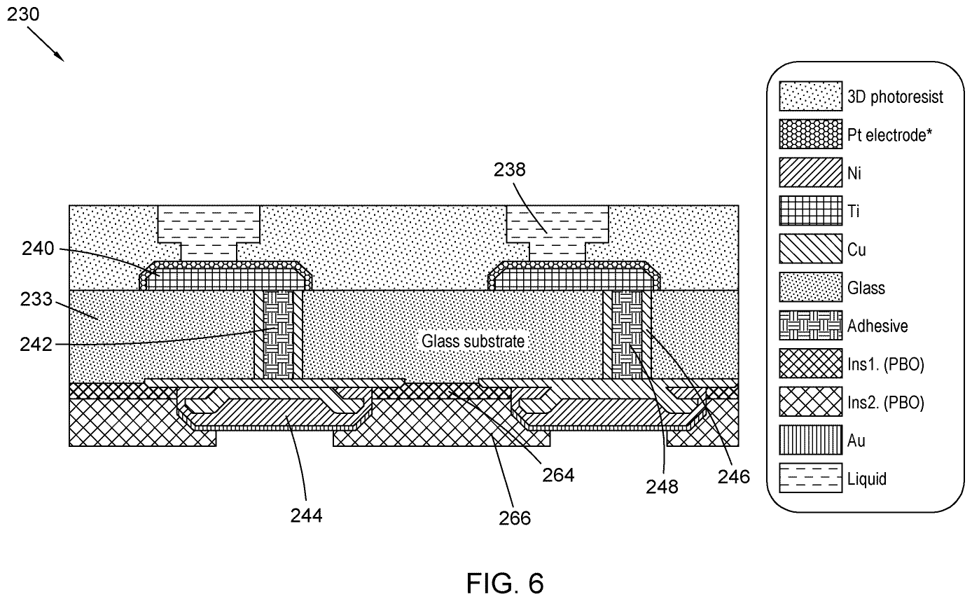

FIG. 6 shows an embodiment of a sensor device 230, similar to the embodiment shown in FIG. 4. The sensor device 230 shown in FIG. 6 a very similar configuration to the sensor device shown in FIG. 4. The sensor device 230 shown in FIG. 6 comprises a via 242 passing through the substrate 233. The via 242 comprises a copper barrel coating 246 that is filled with adhesive 248, but the via 242 does not have a copper cap.

Another difference between the sensor device 230 shown in FIG. 6 and the sensor device shown in FIG. 4 is the shape of the sensor electrode 240 on the front side of the substrate 233. In the sensor device 230 shown in FIG. 6, the sensor electrode 240 has a non-circular shape that is substantially an oval with straight sides.

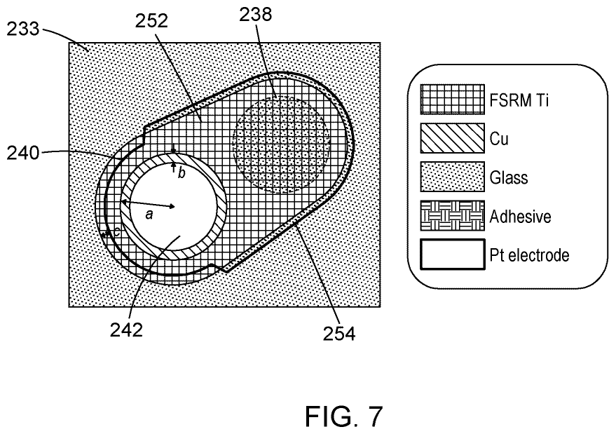

Furthermore, the area of the sensor electrode 240 at the base of the well 238 is offset from its respective via 242. Thus, each well 238 is offset from its respective via 242. The shape and position of the sensor electrode 240 will be described in more detail with reference to FIG. 7.

The other main difference between the sensor device 230 shown in FIG. 6 and the sensor device shown in FIG. 4 is that a second PBO insulation layer 266 is provided over the first PBO insulation layer 264 and the edges of the substrate contact 244 (to thus leave an area of the substrate contact 244 exposed for electrical contact to a ball of solder, for example).

FIG. 7 shows a plan view of the sensor electrode 240 shown in FIG. 6. From this, the relative positioning (and offsets) of the sensor electrode 240 on the substrate 233, the via 242 and the well 238 can be seen. Overall, the sensor electrode 240 has a substantially straight-sided oval shape; however, the platinum coating 254 does not have exactly the same shape as the titanium base layer 252. In the vicinity of (underneath and around) the well 238, the platinum coating 254 extends beyond the boundary of the titanium base layer 252. In the vicinity of (above and around) the via 242, the platinum coating 254 does not extend to the boundary of the titanium base layer 252.

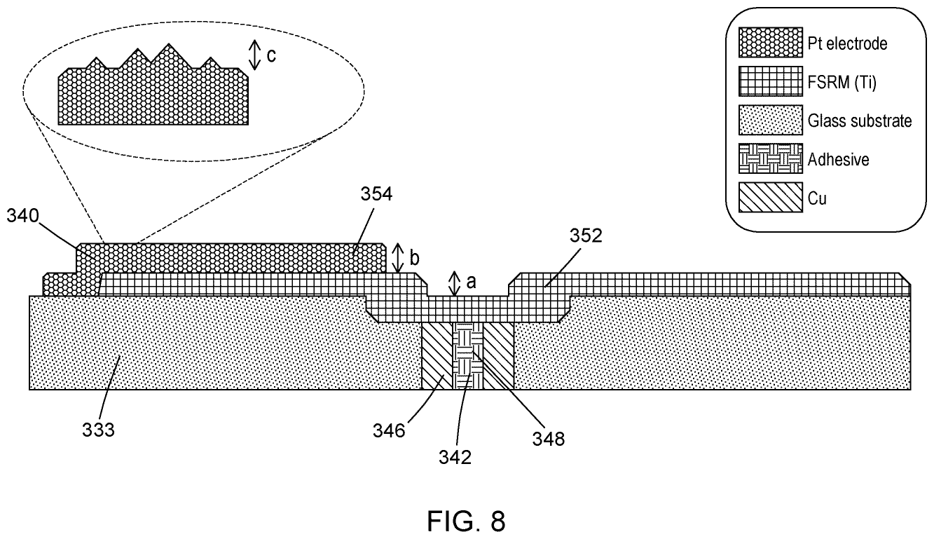

FIG. 8 shows an embodiment of a sensor electrode 340 on the front side of a substrate 333, similar to the embodiment shown in FIGS. 6 and 7. Only the upper half (showing the front side) of the substrate 333 is shown in FIG. 8.

In this embodiment, in which the sensor electrode 340 is offset from the via 342, the via 342 comprises a copper barrel coating 346 that is filled with adhesive 348, but the via 342 does not have a copper cap. Instead, the front side of the substrate 333 surrounding the via 342 is recessed and the titanium base layer 352 of the sensor electrode 340 is formed in and around the recess. The platinum coating 354 (above which a well of a sensing element will be located) does not fully cover the titanium base layer 352 and is only provided on part of the titanium base layer 352, offset from and not covering the titanium base layer 352 above the via 342.

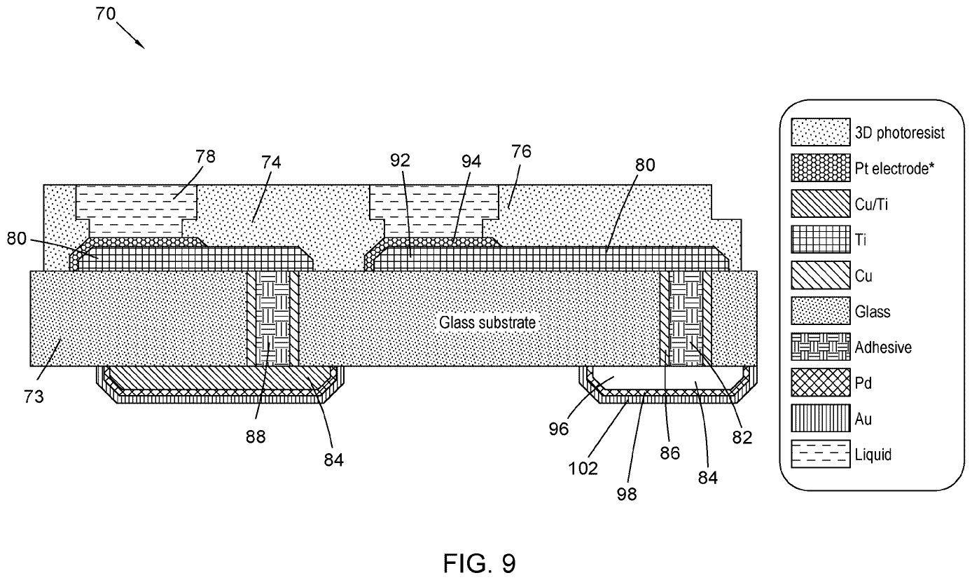

FIG. 9 shows schematically in cross-section a sensor device 70 in accordance with another embodiment of the present invention. The sensor device 70 may, for example, be used in nanopore sensor, for example similar to the one shown in FIG. 3.

Similarly to the sensor devices shown in FIGS. 3 and 4, the sensor device 70 comprises a glass substrate 73 on which is formed a photoresist support structure 74 having a plurality of walls 76 that define between them a plurality of wells 78. The support structure 74 is configured to support a nanopore membrane over each of the wells 78.

A sensor electrode 80 is formed on the front side of the substrate 73 at the bottom of each well 78. A plurality of vias 82 are formed through the substrate 73 to connect the respective plurality of sensor electrodes 80 to a respective plurality of substrate contacts 84 on the back side of the substrate 73.

The vias 82 through the substrate 73 each comprise a copper barrel coating 86 that is filled with adhesive 88 to hermetically seal the vias 82. The vias 82 each have a diameter of approximately 70 μm. In the embodiment shown in FIG. 7, the pitch between the vias 82 (and between the plurality of substrate contacts 84) is approximately 800 μm, while the pitch between the wells 78 is approximately 200 μm.

The sensor electrodes 80 on the front side of the substrate 73 are formed of two layers: a base layer 92 made of titanium (which is arranged to route from the via 82 to the respective well 78, to map the vias 82 at a pitch of approximately 800 μm to the pitch of the sensor electrodes 80 at approximately 200 μm) and a coating 94 made of platinum.

The vias 82 are offset from (and not coaxial with) the centre of the respective wells 78. Each sensor electrode 80 extends between a via 82 and a respective offset well 78. The platinum coating 94 on each sensor electrode 80 does not cover the whole of the titanium base layer 92, with the platinum coating 94 covering the titanium base layer 92 at least partially (for example fully) over the bottom of the well 78 and the immediately surrounding area beneath the photoresist support structure 74. Thus, in some embodiments (having a configuration that differs from that shown in FIG. 9), the fluid in the well may be exposed to the substrate, for example around the edges of the sensor electrode 80,. The platinum coating 94 does not extend to cover the part of the titanium base layer 92 above, and that contacts with, the via 82.

The substrate contacts 84 on the back side of the substrate 73 are each formed of two or three layers: a base layer 96 made of copper or titanium, an optional intermediate layer 98 made of palladium, and a coating 102 made of gold. The intermediate layer 98 and the coating 102 may be applied to the base layer 96 using an electroless palladium immersion gold (EPIG) finish. The copper or titanium base layer 96 connects to, and extends on the substrate 73 over and around, the barrel coating 86 in the via 82.

The substrate contact 84 may be centred on its respective via 82 (as shown for the substrate contact 84 on the right hand side of FIG. 7) or the substrate contact 84 may be offset from its respective via 82 (as shown for the substrate contact 84 on the left hand side of FIG. 7).

The substrate contacts 84 on the back side of the substrate 73 are configured for connection to input contacts of a PCB by respective spring contacts.

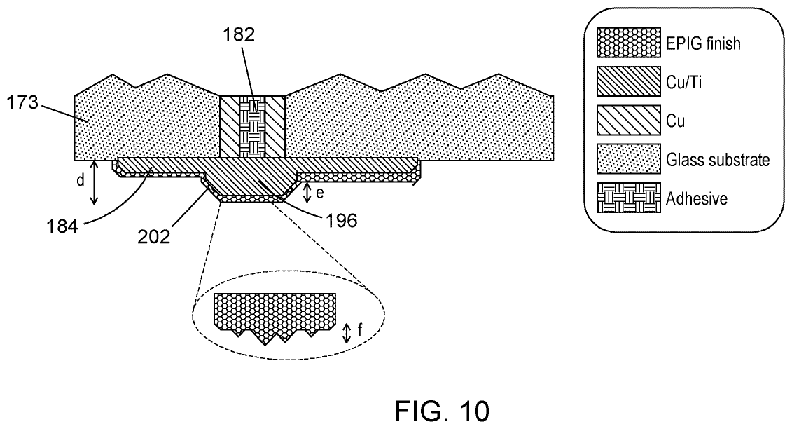

FIG. 10 shows an embodiment of a substrate contact 184 on the back side of a substrate 173, similar to the embodiment shown in FIG. 9. Only the lower half (showing the back side) of the substrate 173 is shown in FIG. 8.

In this embodiment, the copper or titanium base layer 196 of the substrate contact 184 is raised in the centre of the substrate contact 184 (in the vicinity of the via 182) and thus projects from the surrounding area of the substrate contact 184. The gold coating 202 of the substrate contact 184 extends over the whole area of the substrate contact 184.

The projection on the substrate contact 184 helps to engage with the respective spring contact from the PCB, thus helping to form a good electrical connection.

1. A sensor device for a nanopore sensor, the sensor device comprising:
an insulating substrate;
one or more wells for containing a fluid;
wherein the one or more wells are formed on a first side of the substrate;
a sensor electrode for detecting an ionic current in each of the one or more wells;
wherein the sensor electrodes are formed on the first side of the substrate at the base of the one or more wells;
one or more contacts formed on a second side of the substrate; and
one or more vias extending through the substrate;
wherein the one or more vias connect the sensor electrodes to the one or more contacts;
wherein the electrodes comprise a different structure from the one or more contacts and/or the sensor electrodes are made from a different material or materials from the one or more contacts.

2. The sensor device as claimed in claim 1, wherein the insulating substrate comprises a glass substrate.

3. The sensor device as claimed in claim 1 or 2, wherein each of the sensor electrodes comprises an electrode base layer proximal to the substrate and an electrode coating exposed to the well.

4. The sensor device as claimed in claim 3, wherein the electrode base layer comprises a transition metal, for example titanium.

5. The sensor device as claimed in claim 3 or 4, wherein the electrode coating comprises a noble metal, for example platinum.

6. The sensor device as claimed in claim 3, 4 or 5, wherein the electrode coating has a greater electrical conductivity than the electrode base layer.

7. The sensor device as claimed in any one of claims 3 to 6, wherein the electrode coating is formed from a less reactive material than the electrode base layer.

8. The sensor device as claimed in any one of the preceding claims, wherein each of the one or more contacts comprises a contact base layer proximal to the substrate and a contact outer coating.

9. The sensor device as claimed in claim 8, wherein the contact base layer comprises a transition metal, for example copper.

10. The sensor device as claimed in claim 8 or 9, wherein the contact outer coating comprises a noble metal, for example gold.

11. The sensor device as claimed in claim 8, 9 or 10, wherein the contact outer coating has a greater electrical conductivity than the contact base layer.

12. The sensor device as claimed in any one of claims 8 to 11, wherein each of the one or more contacts comprises a contact intermediate layer between the contact base layer and the contact outer coating.

13. The sensor device as claimed in claim 12, wherein the contact intermediate layer comprises a transition metal, for example nickel.

14. The sensor device as claimed in claim 12 or 13, wherein the contact outer coating has a greater electrical conductivity than the contact intermediate layer 15. The sensor device as claimed in any one of the preceding claims, wherein the sensor device comprises an insulating layer on the second side of the substrate surrounding the one or more contacts.

16. The sensor device as claimed in any one of the preceding claims,
wherein the one or more vias each comprise:
a conductor extending through the via; and
a material arranged to retain the conductor in the via;
wherein the material arranged to retain the conductor in the via is different from a material of the conductor.

17. The sensor device as claimed in claim 16, wherein the material arranged to retain the conductor in the via substantially fills the via.

18. The sensor device as claimed in claim 16 or 17, wherein the material arranged to retain the conductor in the via comprises a sealant, for example an adhesive.

19. The sensor device as claimed in claim 16, 17 or 18, wherein the conductor comprises a conducting barrel that at least partially lines the via.

20. The sensor device as claimed in claim 19, wherein the via is defined by a wall through the substrate; and
wherein the conducting barrel at least partially lines the wall of the via.

21. The sensor device as claimed in claim 19 or 20, wherein the material arranged to retain the conductor in the via substantially fills the conducting barrel.

22. The sensor device as claimed in claim 19, 20 or 21, wherein the material arranged to retain the conductor in the via is arranged to substantially seal the conducting barrel in the via.

23. The sensor device as claimed in any one of the preceding claims,
wherein the one or more vias each comprise a conducting cap at one or both ends of the via.

24. The sensor device as claimed in claim 23, wherein the one or more vias each comprise a conducting cap at both ends of the via.

25. The sensor device as claimed in any one of the previous claims wherein each wells support a membrane containing a nanopore channel inserted in the membrane.

26. The sensor device as claimed in claim 25 wherein the membrane comprises amphiphilic molecules such as a lipid or a polymer.
